(19) United States
(12) Patent Application Publication
(10) Pub. No.: US 2010/0137751 A1
(43) Pub. Date: Jun. 3, 2010

(54) ULTRASONIC OPERATION APPARATUS

(76) Inventor: Yusuke Tadami, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/326,155

(22) Filed: Dec. 2, 2008

(51) Int. Cl. A61N 7/00; A61B 18/14

(52) U.S. Cl. 601/2; 606/41

(57) ABSTRACT

An ultrasonic surgical instrument that comprises a handpiece configured to treat living tissue with at least one of an ultrasonic vibration and a high-frequency current, a first generation unit configured to generate a drive current for producing the ultrasonic vibration, a second generation unit configured to generate the high-frequency current, a suction device configured to draw mist produced at the living tissue when the handpiece treats the living tissue with the ultrasonic vibration, and a control unit configured to control the suction device, causing the section device to draw the mist when outputs of both the first generation unit and the second generation unit are detected or when only the output of the first generation unit is detected. The apparatus further comprises a suction tube connected to the suction device, a suction path communicating with the suction tube, and a suction port made in the suction path.

### BACKGROUND OF THE INVENTION

1. Field of the Invention

The present invention relates to an ultrasonic surgical instrument that can draw mist produced by fatty tissue or the like that is undergoing ultrasonic vibration.

2. Description of the Related Art

In most ultrasonic surgical instruments, the drive current is supplied from the power supply device to the ultrasonic transducer provided in the handpiece. A probe is attached to the ultrasonic transducer. At the distal end of the probe, a surgical instrument is provided. The ultrasonic transducer has a piezoelectric element. The piezoelectric element converts the drive current into ultrasonic vibration. The ultrasonic vibration is transmitted from the ultrasonic transducer via the probe to the surgical instrument. The ultrasonic vibration is applied to tissue (e.g., fatty tissue), to treat the tissue.

Such an ultrasonic surgical instrument also supplies a high-frequency current to tissue on which to treat. The ultrasonic surgical instrument can treat the tissue with the high-frequency current. If the drive current and the high-frequency current are supplied to the tissue at the same time, the tissue will be efficiently treated with the high-frequency current, while undergoing the ultrasonic vibration produced by the drive current.

Jpn. Pat. Appln. KOKAI Publication No. 6-343647, for example, discloses an ultrasonic surgical instrument that can supply a high-frequency current and excels in operability.

Jpn. Pat. Appln. KOKAI Publication No. 2002-306507, for example, discloses a surgical instrument and a method of preventing foreign matter from adhering to a surgical instrument.

### BRIEF SUMMARY OF THE INVENTION

The present invention provides an ultrasonic surgical instrument that can efficiently treat tissue, such as fatty tissue, causing no bleeding at the tissue, can prevent mist from impairing the view field at the tissue, can prevent the mist from flashing, and can output an ultrasonic vibration and a high-frequency current at the same time.

The invention provides an ultrasonic surgical instrument that comprises: a handpiece configured to treat living tissue with at least one of an ultrasonic vibration and a high-frequency current; a first generation unit configured to generate a drive current for producing the ultrasonic vibration; a second generation unit configured to generate the high-frequency current; a suction device configured to draw mist produced at the living tissue when the handpiece treats the living tissue with the ultrasonic vibration; a control unit configured to control the suction device, causing the section device to draw the mist when outputs of both the first generation unit and the second generation unit are detected or when only the output of the first generation unit is detected; a suction tube connected to the suction device; a suction path connected at a proximal end to the suction tube and configured to guide the mist drawn by the suction device; and a suction port provided in a distal end of the suction path and configured to draw the mist into the suction path.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing an ultrasonic surgical instrument according to a first embodiment of the present invention;

FIG. 2 is a sectional view of the handpiece of the ultrasonic surgical instrument;

FIG. 3 is a flowchart explaining how the first embodiment may operate;

FIG. 4A is a timing chart showing when the high-frequency current generation unit, ultrasonic vibration generation unit and suction device produce outputs while the ultrasonic surgical instrument is operating in the first mode;

FIG. 4B is a timing chart showing when the ultrasonic vibration generation unit and suction device produce outputs while the ultrasonic surgical instrument is operating in the second mode;

FIG. 5 is a schematic diagram showing an ultrasonic surgical instrument according to a second embodiment of the invention;

FIG. 6 is a sectional view of a handpiece of a third embodiment of the invention;

FIG. 7 is a sectional view of a handpiece of a fourth embodiment of the invention;

FIG. 8A is a magnified view of the distal end of the probe of a fifth embodiment of the invention;

FIG. 8B is another magnified view of the distal end of the probe of a fifth embodiment of the invention;

FIG. 8C is a sectional view taken along line A-A shown in FIGS. 8A and 8B;

FIG. 8D is a sectional view taken along line B-B shown in FIGS. 8A and 8B;

FIG. 9A is a schematic diagram showing an ultrasonic surgical instrument according to a sixth embodiment of the invention;

FIG. 9B is a flowchart explaining how the sixth embodiment operates;

FIG. 10A is a schematic diagram showing an ultrasonic surgical instrument according to a seventh embodiment of the invention;

FIG. 10B is a part of a flowchart explaining how the seventh embodiment operates; and

FIG. 10C is the remaining part of the flowchart explaining how the seventh embodiment operates.

### DETAILED DESCRIPTION OF THE INVENTION

Embodiments of this invention will be described in detail, with reference to the accompanying drawings.

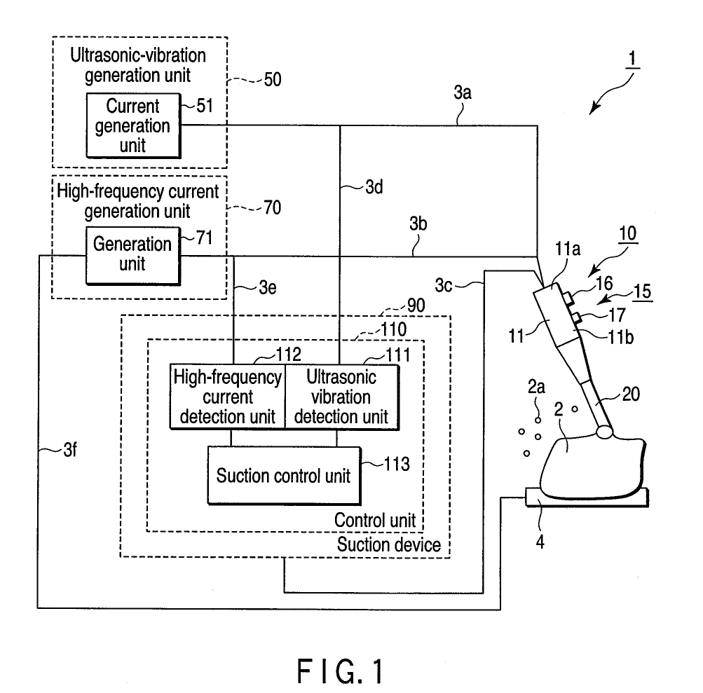

A first embodiment will be described with reference to FIG. 1 to 4.

As FIG. 1 shows, an ultrasonic surgical instrument 1 for use in surgery has a handpiece 10, an ultrasonic-vibration generation unit 50, a high-frequency current generation unit 70, a suction device 90, and a control unit 110. The handpiece 10 uses ultrasonic vibration or a high-frequency current, or both, to treat tissue 2 present in, for example, the body cavity. The ultrasonic-vibration generation unit 50 generates a drive current for producing ultrasonic vibration. The high-frequency current generation unit 70 generates a high-frequency current. The suction device 90 can draw mist 2a rising from the tissue 2 undergoing the ultrasonic vibration while being treated with the handpiece 10. The control unit 110 controls the suction device 90, causing the suction device 90 to draw the mist 2a, upon detecting both the output of the ultrasonic-vibration generation unit 50 and the output of the high-frequency current generation unit 70, or only the output of the ultrasonic-vibration generation unit 50.

The ultrasonic surgical instrument 1 further has a suction tube 3c, a suction path 13, and a suction port 13c. The suction tube 3c is connected to the suction device 90. The suction path 13 communicates, at proximal end 13a, with the suction tube 3c, for guiding mist 2a drawn into the suction tube 3c. The suction port 13c is provided at the distal end 13b of the suction path 13, for drawing the mist 2a into the suction path 13.

The suction device 90 incorporates the control unit 110.

Note that the tissue 2 on which to treat is, for example, fatty tissue. To “treat” is to apply, for example, ultrasonic vibration or a high-frequency current to the tissue.

The ultrasonic surgical instrument 1 has a footswitch (not shown), which may be operated to control the ultrasonic-vibration generation unit 50, high-frequency current generation unit 70 and suction device 90 and to setting the unit 50, unit 70 and device 90 in desired states. A cable (not shown) connects the footswitch to the handpiece 10.

The handpiece 10 is connected to the ultrasonic-vibration generation unit 50 by a cable 3a, to the high-frequency current generation unit 70 by a cable 3b, and to the suction device 90 by the suction tube 3c. The ultrasonic-vibration generation unit 50 is connected to the control unit 110 by a cable 3d. The high-frequency current generation unit 70 is connected to the control unit 110 by a cable 3e. The unit 70 is also connected by a cable 3f to a counterelectrode 4 that is positioned in contact with the tissue 2.

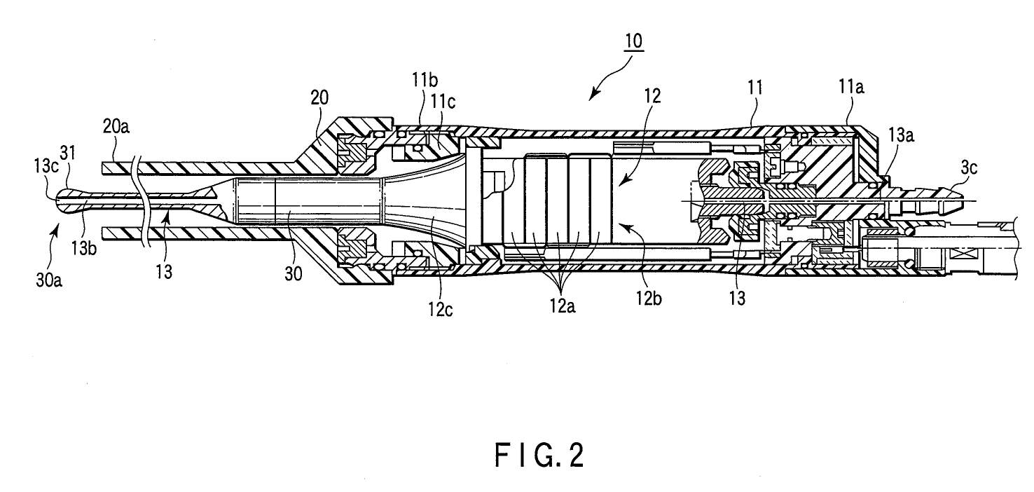

As shown in FIG. 2, the handpiece 10 has a hollow cylindrical case 11 at the proximal part. The cables 3a and 3b (not shown in FIG. 2) and the suction tube 3c (not shown in FIG. 2) are connected to the proximal end 11a of the case 11. A sheath 20, in which a probe 30 is inserted, is connected to the distal end 11b of the case 11. (The probe 30 is a transmission member that will be described later.) In other words, the sheath 20 covers the probe 30 and is coupled to the distal end 11b of the case 11.

In the proximal end of the case 11, an ultrasonic transducer unit 12 is provided. The unit 12 is a hollow member that produces ultrasonic vibration. The ultrasonic transducer unit 12 is secured in the case 11 means of a fastening member 11c. The ultrasonic transducer unit 12 converts the drive current generated by the ultrasonic-vibration generation unit 50 and supplied via the cable 3a into ultrasonic vibration.

The ultrasonic transducer unit 12 has an ultrasonic transducer 12b and a horn 12c. The ultrasonic transducer 12b is configured to produce ultrasonic vibration (e.g., vertical ultrasonic vibration). The horn 12c is arranged in front of the ultrasonic transducer 12b, as viewed in the longitudinal direction of the case 11 (probe 30). The horn 12c can amplify the ultrasonic vibration produced by the ultrasonic transducer 12b.

The ultrasonic transducer 12b comprises a plurality of ring-shaped piezoelectric elements 12a. The piezoelectric elements 12a are densely arranged side by side, extending in the longitudinal direction of the case 11. The piezoelectric element 12a located most distal is connected to the horn 12c. Each piezoelectric element 12a is a member that converts a drive current (electric power) to ultrasonic vibration. The drive current has been generated by the ultrasonic-vibration generation unit 50 and supplied from the unit 50 via the cable 3a to the piezoelectric elements 12a. The drive current is converted to ultrasonic vibration by the piezoelectric elements 12a.

The ultrasonic transducer 12b is secured in the proximal end of the case 11. The ultrasonic transducer 12b is, for example, a bolt-clamped Langevin type transducer (BLT).

The horn 12c is made of metal such as titanium, duralmin, or stainless steel.

The probe 30 is attached to the horn 12c, extending through the sheath 20. The probe 30 is a transmission member that receives the ultrasonic vibration amplified by the horn 12c and transmits the vibration to the distal end of the handpiece 10. The distal end 30a of the probe 30 projects from the distal end 20a of the sheath 20. The ultrasonic vibration produced by the ultrasonic transducer 12b and transmitted by the probe 30 is applied to a surgical element 31. Using the surgical element 31 vibrated with the ultrasonic vibration, the surgeon may treat the tissue 2. The surgical element 31 is arranged at the distal end 30a and configured to treat the tissue 2 with the ultrasonic vibration produced by the ultrasonic transducer 12b and transmitted from the probe 30.

The probe 30 is thus arranged, extending through the sheath 20. The probe 30 is connected, at proximal end, to the ultrasonic transducer 12b by the horn 12c. The probe 30 can therefore transmit the ultrasonic vibration produced by the ultrasonic transducer 12b, from the proximal end of the probe 30 to the distal end 30a of the probe 30 (thence to the surgical element 31 and the distal end of the handpiece 10). The surgical element 31 can therefore serve to treat the tissue 2.

The surgical element 31 can treat the tissue, also with the high-frequency current generated by the high-frequency current generation unit 70 and supplied through the cable 3b to the ultrasonic transducer 12b and probe 30. The high-frequency current flows from the surgical element 31 to the tissue 2 and thence to the counterelectrode 4. This current then flows back to the high-frequency current generation unit 70 through the cable 3f.

In this embodiment, the surgical element 31 of the handpiece 10 treats the tissue 2, using the ultrasonic vibration and the high-frequency current at the same time. Alternatively, the surgical element 31 may treat the tissue 2, using only the ultrasonic vibration.

The probe 30 is removably fastened to the horn 12c with, for example, screws. Like the horn 12c, the probe 30 is made of metal such as titanium, duralmin, or stainless steel.

The suction path 13 extends through the ultrasonic transducer unit 12 and probe 30, in the longitudinal direction of the handpiece 10. Thus, the suction path 13 connects the ultrasonic transducer unit 12 and the probe 30. The ultrasonic transducer unit 12 and the probe 30 therefore communicate with each other, extending in the longitudinal direction of the handpiece 10. The suction path 13 is a passage through which the suction device 90 can draw mist 2a. The suction path 13 communicates, at proximal end 13a, with the suction tube 3c. The distal end 13b of the suction path 13 is arranged in the surgical element 31 (more precisely, distal end 30a). The distal end 13b (surgical element 31) has a suction port 13c. When the suction device 90 is driven, the mist 2a is drawn into the suction port 13c and flows into the suction tube 3c through the suction path 13.

As shown in FIG. 1, a handswitch unit 15 is provided on the case 11. When operated, the handswitch unit 15 can turn on and off the ultrasonic-vibration generation unit 50 and the high-frequency current generation unit 70. The handswitch unit 15 has a first switch 16 and a second switch 17.

The first switch 16 controls the ultrasonic-vibration generation unit 50 and the high-frequency current generation unit 70 via the cables 3a and 3b. That is, the first switch 16 changes the surgical mode of the handpiece 10, to the first mode in which the tissue 2 is treated with both the ultrasonic vibration and the high-frequency current at the same time.

The second switch 17 controls only the ultrasonic-vibration generation unit 50 via the cable 3a. That is, the second switch 17 changes the surgical mode of the handpiece 10, to the second mode in which the tissue 2 is treated with the ultrasonic vibration only.

Thus, the case 11 contains the handswitch unit 15 that may be operated to change the surgical mode of the handpiece 10 to the first mode in which the tissue 2 is treated with both the ultrasonic vibration and the high-frequency current at the same time, or to the second mode in which the tissue 2 is treated with the ultrasonic vibration only.

The handswitch unit 15 may have another switch (not shown), which controls the high-frequency current generation unit 70 via the cable 3b. When operated, this switch changes the surgical mode of the handpiece 10 to the third mode in which the tissue 2 is treated with the high-frequency current only.

The ultrasonic-vibration generation unit 50 has a current generation unit 51. The current generation unit 51 generates, when the first switch 16 or the second switch 2 is closed, a drive current for producing ultrasonic vibration. The current generation unit 51 can control the amplitude of the ultrasonic vibration.

The high-frequency current generation unit 70 has a generation unit 71. The generation unit 71 produces ultrasonic vibration as long as the first switch 16 remains on.

The control unit 110 has an ultrasonic vibration detection unit 111, a high-frequency current detection unit 112 and a suction control unit 113.

The ultrasonic vibration detection unit 111 is connected by the cable 3d to the current generation unit 51. The ultrasonic vibration detection unit 111 detects whether the ultrasonic-vibration generation unit 50 has generated the drive current (or ultrasonic vibration). That is, the unit 111 can determines whether the ultrasonic-vibration generation unit 50 is outputting the drive current.

The high-frequency current detection unit 112 is connected by the cable 3e to the generation unit 71. The high-frequency current detection unit 112 detects whether the high-frequency current generation unit 70 has generated a high-frequency current. That is, the unit 112 can determines whether the high-frequency current generation unit 70 is outputting the high-frequency current.

The suction control unit 113 controls the suction device 90 to cause the suction device 90 to draw the mist 2a when the ultrasonic vibration detection unit 111 detects the output of drive current. To “detect the output of drive current” is to detect that the ultrasonic-vibration generation unit 50 has output the drive current and that the surgical mode has been changed to the first mode or the second mode.

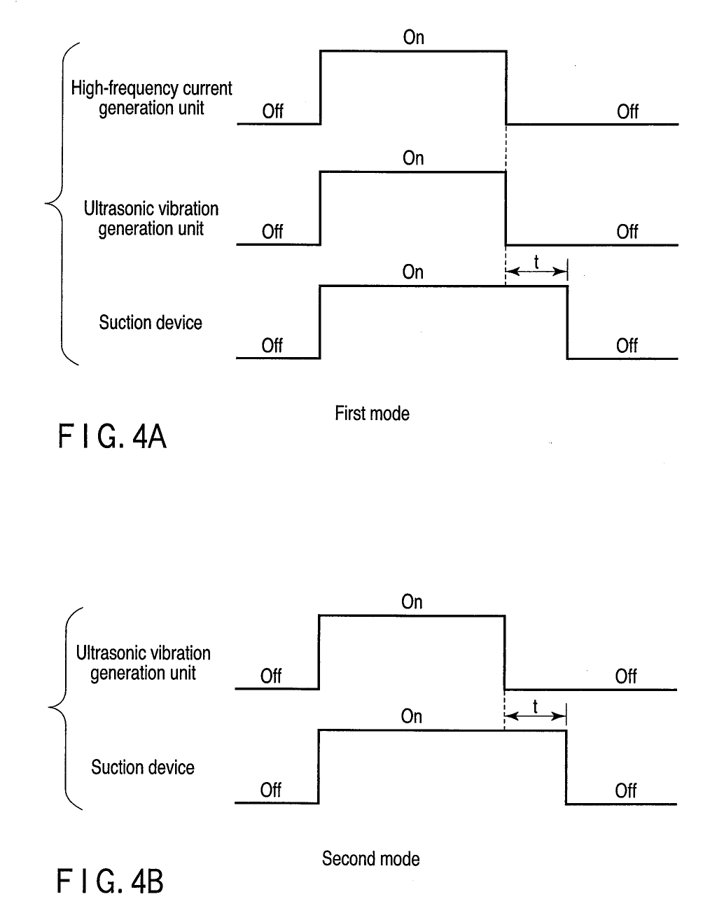

After the ultrasonic-vibration generation unit 50 has stopped operating, the suction control unit 113 controls the suction device 90, causing the suction device 90 to draw the mist 2a for desirable time t as shown in FIGS. 4A and 4B. Desirable time t is a period starting when the ultrasonic-vibration generation unit 50 stops operating.

That is, the suction control unit 113 makes the suction device 90 keep drawing the mist 2a for time t as illustrated in FIG. 4A, after the tissue 2 has been treated with both the ultrasonic vibration and the high-frequency current (in the first mode) or treated with the ultrasonic vibration only (in the second mode). The completion of the surgery using the ultrasonic vibration and the high-frequency current (in the first mode) means two events. The first event is that both the ultrasonic-vibration generation unit 50 and the high-frequency current generation unit 70 have stopped operating. The second event is that the ultrasonic vibration detection unit 111 and the high-frequency current detection unit 112 have detected that the drive current and the high-frequency current are no longer output. Further, the completion of the surgery using the ultrasonic vibration only (in the second mode) means that the ultrasonic-vibration generation unit 50 has stopped operating and that the ultrasonic vibration detection unit 111 has detected that the drive current is no longer supplied.

In other words, the suction control unit 113 controls the suction device 90, causing the suction device 90 to draw the mist 2a for desirable time t, if at least one of the output of the ultrasonic wave and the output of high-frequency current is detected after the ultrasonic-vibration generation unit 50 and the high-frequency current generation unit 70 have stop operating at the same time as illustrated in FIG. 4A and FIG. 4B.

FIG. 4B is a timing chart showing when the ultrasonic vibration generation unit 50 and suction device 90 produce outputs. FIG. 4B is also a diagram showing when the high-frequency current generation unit 70 and suction device 90 produce outputs.

That the ultrasonic-vibration generation unit 50 and the high-frequency current generation unit 70 “have stopped operating” also means that the ultrasonic-vibration generation unit 50 and the high-frequency current generation unit 70 “have stopped operating at the same time.”

If the surgery with at least one of the ultrasonic wave and the high-frequency current is performed, the suction device 90 starts drawing the mist 2a at the same time the surgery is started, as is illustrated in FIG. 4A and FIG. 4B. When the surgery with at least one of ultrasonic wave and the high-frequency current is completed, the suction device 90 draws the mist 2a for desirable time t.

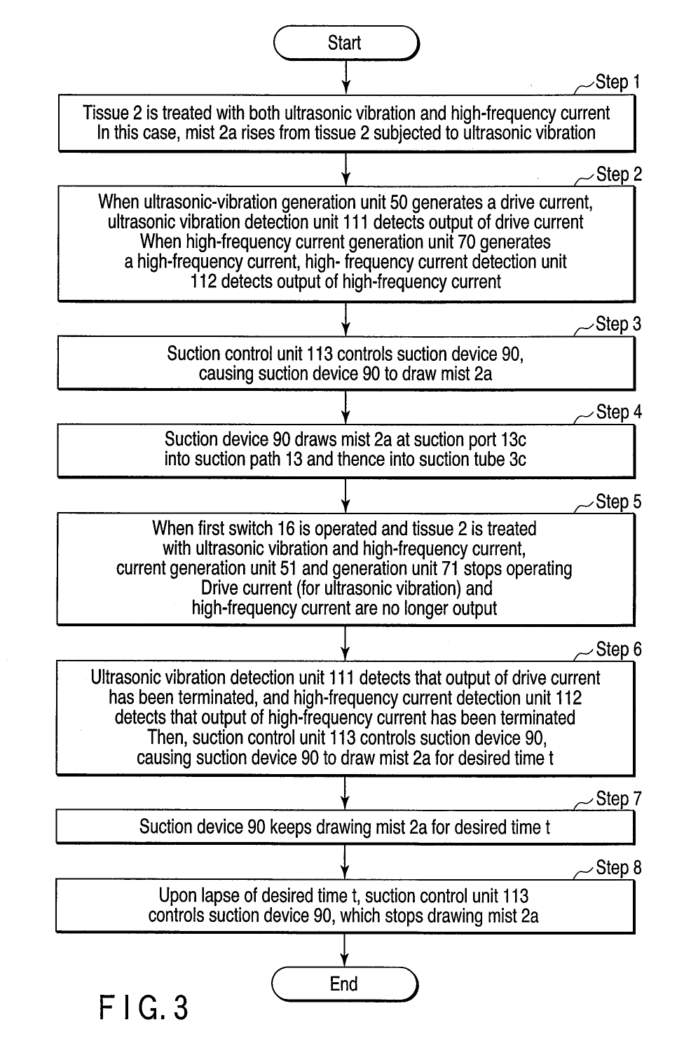

How this embodiment operates will be explained with reference to FIG. 3.

Assume that the surgeon sets the surgical element 31 into contact the tissue 2 and then operates the first switch 16. The surgical mode of the handpiece 10 is thereby changed to the first mode.

As a result, the current generation unit 51 generates a drive current to produce ultrasonic vibration. The drive current is supplied through the cable 3a to the piezoelectric elements 12a. The piezoelectric elements 12a converts the drive current to ultrasonic vibration. The ultrasonic vibration is amplified by the horn 12c. The vibration thus amplified is transmitted from the horn 12c via the probe 30 to the surgical element 31. The tissue 2 is therefore treated with the ultrasonic vibration. At this point, the tissue 2 produces mist 2a because of the ultrasonic vibration.

In the first mode, the generation unit 71 generates a high-frequency current at the same time the current generation unit 51 generates the drive current. The high-frequency current is supplied through the cable 3b to the piezoelectric elements 12a, and thence to the surgical element 31 via the horn 12c and probe 30. Hence, the tissue 2 is also thereby treated with the high-frequency current. After flowing through the tissue 2, the high-frequency current flows to the counterelectrode 4 and then flows back to the high-frequency current generation unit 70 through the cable 3f.

The tissue 2 is thus treated with both the ultrasonic vibration and the high-frequency current. In this case, mist 2a rises from the tissue 2 that subjected to ultrasonic vibration (Step 1).

When the ultrasonic-vibration generation unit 50 generates a drive current, the ultrasonic vibration detection unit 111 detects the output of drive current, and when high-frequency current generation unit 70 generates a high-frequency current, the high-frequency current detection unit 112 detects the output of high-frequency current (Step 2).

If the ultrasonic vibration detection unit 111 and the high-frequency current detection unit 112 detect the output of drive current and the output of high-frequency current, respectively, the suction control unit 113 controls the suction device 90, causing the suction device 90 to draw the mist 2a (Step 3).

That is, the suction device 90 draws the mist 2a at the suction port 13c into the suction path 13 and thence into the suction tube 3c (Step 4).

When the first switch 16 is operated and the tissue 2 is treated with the ultrasonic vibration and the high-frequency current, the current generation unit 51 and generation unit 71 stops operating. The drive current (for ultrasonic vibration) and the high-frequency current are no longer output (Step 5).

At this point, the ultrasonic vibration detection unit 111 detects that the output of drive current has been terminated, and the high-frequency current detection unit 112 detects that the output of high-frequency current has been terminated. Then, the suction control unit 113 controls the suction device 90, causing the suction device 90 to draw the mist 2a for desired time t (Step 6).

Thus, the suction device 90 keeps drawing the mist 2a for desired time t (Step 7).

Upon lapse of desired time t, the suction control unit 113 controls the suction device 90, which stops drawing the mist 2a. The operation of the embodiment is thus terminated (Step 8).

The surgical mode of the handpiece 10 may be changed to the second mode (to treat the tissue 2 with the ultrasonic vibration only). In this case, the suction device 90 draws the mist 2a as shown in FIG. 4B, in the same way as in the first mode. After the tissue 2 has been treated, the suction device 90 draws the mist 2a for desired time t as shown in FIG. 4B, in the same way as in the first mode.

If the tissue 2 full of blood is treated with the high-frequency current only, a coagulated layer will be formed on the tissue 2 as in most cases. Such a coagulated layer adheres to the probe 30 if the probe 30 contacts the tissue 2 in order to treat the tissue 2. The coagulated layer exfoliates from the tissue 2 as the probe 30 is moved away from the tissue 2. As a result, the tissue 2 may bleed.

The tissue 2 will not bleed if it is treated with the ultrasonic vibration and the high-frequency current at the same time. The principle of this non-bleeding surgery resides in that although a coagulated layer is formed as the high-frequency current is supplied to the tissue 2, the ultrasonic vibration prevents the coagulated layer from adhering to the probe 30. More precisely, the coagulated layer will not exfoliate from the tissue 2 when the probe 30 is moved from the tissue 2.

Thus, when both the ultrasonic vibration and the high-frequency current are used, the function of the high-frequency current can be maximized, which is not easy to achieve with ordinary ultrasonic surgical instruments.

The ultrasonic surgical instrument can supply physiological saline solution to the tissue 2 and can utilize the cavitation effect of ultrasonic vibration to crush, emulsify and draw the tissue 2.

When the tissue 2 is treated, while applying ultrasonic vibration to stop bleeding, however, mist 2a rises from the tissue 2 because of the cavitation, possibly impairing the view field of the apparatus. Further, discharge sparks resulting from the high-frequency current (produced while the tissue 2 is being treated with the high-frequency current) may flash the mist 2a. Should it happen, those parts of the tissue 2, which need not be treated, will be damaged.

To prevent the production of mist 2a, the probe 30 may be so designed to reduce cavitation. The probe 30 can hardly designed so, because it must be so shaped to meet various uses. The cavitation can indeed be reduced by decreasing the amplitude of the ultrasonic vibration, ultimately to prevent the production of mist 2a. However, the coagulated layer will adhere to the probe 30, inevitably lowering the efficiency of stop bleeding at the tissue 2a.

In view of this, the suction device 90 draws the mist 2a away from the tissue 2 at the same time the ultrasonic vibration and the high-frequency current are output. Therefore, this embodiment can enable the surgeon to treat the tissue 2 with high efficiency, without causing bleeding of the tissue 2. In addition, the suction device 90 can draw the mist 2a, irrespective of the shape of the probe 30. The probe 30 does not impair the view field, whatever shape it has. Moreover, the mist 2a can be prevented from flashing due to the high-frequency current. Further, the present embodiment never damages the tissue 2.

In this embodiment, the suction device 90 draws the mist 2a for desired time t after the ultrasonic-vibration generation unit 50 stops generating the drive current. Therefore, the mist 2a can be reliably drawn, not remaining in the body cavity at all.

After the output of the ultrasonic vibration and the output of the high-frequency current have been stopped in this embodiment, the suction device 90 draws the mist 2a for only desired time t in order to prevent the mist 2a from flashing in the body cavity, if at least one of the ultrasonic vibration and the high-frequency current is detected. This reliably draws the mist 2a, and no mist will remain in the body cavity. Moreover, in this embodiment, the mist 2a can be prevented from flashing due to, for example, the high-frequency current, because the suction device 90 draws the mist 2a even if only the output of high-frequency current is detected.

In addition, the surgical mode can be changed to either the first mode or the second mode, merely by operating the first switch 16 or the second switch 17. Thus, for the surgeon, the present embodiment is easy to operate to change the surgical mode. An operator does not have to accustom himself/herself to the operation, and a burden of the operation on the operator can be suppressed.

In this embodiment, the suction path 13 connects the ultrasonic transducer unit 12 and the probe 30. The diameter of the handpiece 10 which draws mist 2a can therefore be more reduced than otherwise.

The suction port 13c is provided at the distal end 13b of the suction path 13 in this embodiment. The mist 2a can therefore be drawn from the vicinity of the surgical element 31, preventing the mist 2a from scattering around.

Further, the amplitude of the ultrasonic vibration can be controlled as desired in the current generation unit 51. The tissue 2 can therefore be prevented from adhering to, or coagulating on, the wall of the body cavity. This enables the surgeon to treat the tissue 2, without causing bleeding.

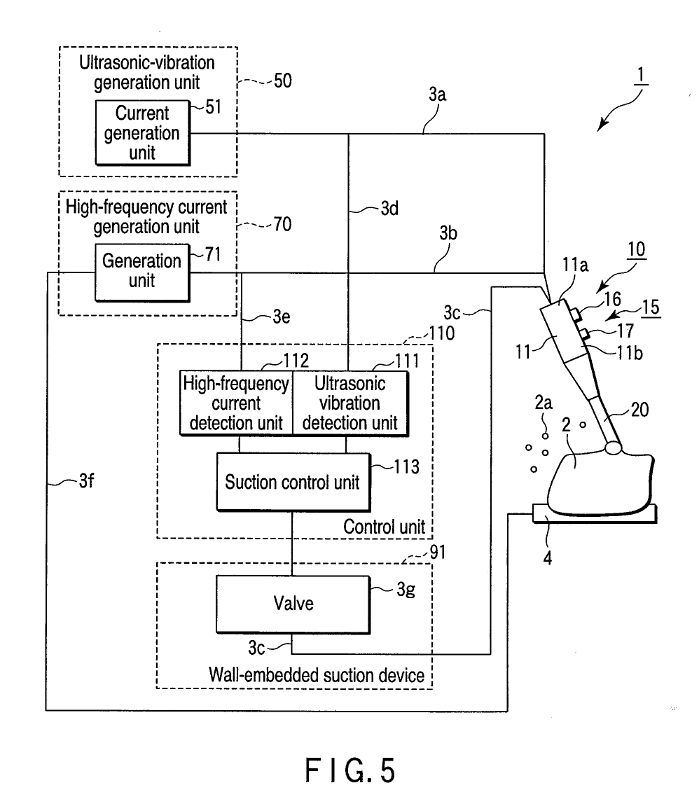

A second embodiment of the present invention will be described with reference to FIG. 5. The components identical to those of the first embodiment are designated by the same reference numbers and will not be described.

The suction device of this embodiment is a wall-embedded suction device 91 of the ordinary type for installation in operation rooms.

A valve 3g that can be opened and closed is provided on the suction tube 3c. When the ultrasonic vibration detection unit 111 detects the output of the drive current in the first or second mode, the suction control unit 113 opens the valve 3g to draw the mist 2a. The suction control unit 113 closes the valve 3g upon lapse of desired time t after the ultrasonic-vibration generation unit 50 stops generating the drive current.

How the present embodiment operate will be explained briefly.

In Step 3, the suction control unit 113 opens the valve 3g and controls the suction device 90, causing the device 90 to draw the mist 2a.

In Step 8, the suction control unit 113 closes the valve 3g upon lapse of desired time t, and controls the suction device 90, causing the suction device 90 to stop operating. As a result, the suction device 90 stops drawing the mist 2a. That is, in this embodiment, the valve 3g mechanically opens the suction path defined by the suction tube 3c to draw the mist 2a, and closes the suction path to stop drawing the mist 2a.

In any other respects, this embodiment is the same as the first embodiment.

As has been pointed out, the suction device of this embodiment is a wall-embedded suction device 91 of the type for installation in operation rooms. Therefore, the suction device need not be incorporated in the ultrasonic surgical instrument 1. Hence, the ultrasonic surgical instrument 1 according to this embodiment can be simplified and can therefore be less expansive.

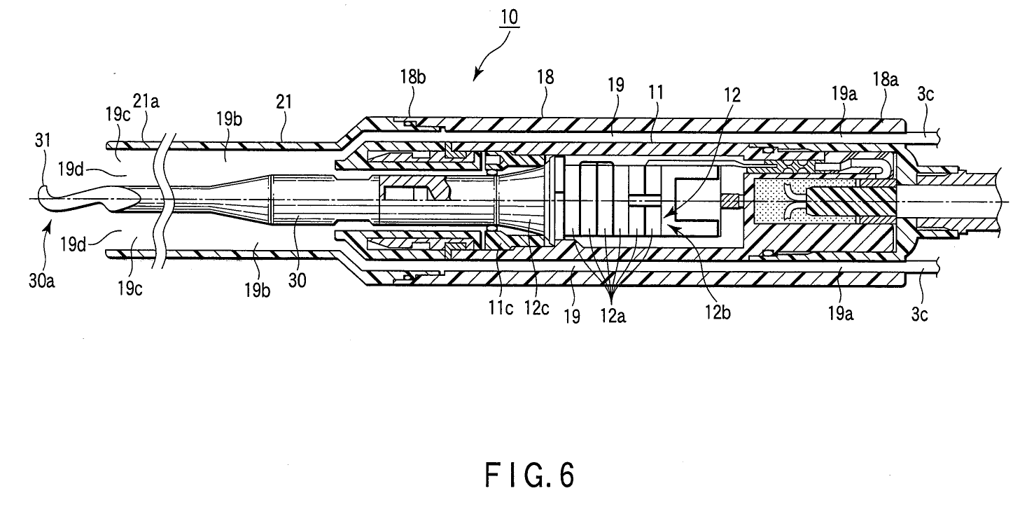

A third embodiment of the present invention will be described with reference to FIG. 6. The components identical to those of the first and second embodiments are designated by the same reference numbers and will not be described.

As shown in FIG. 6, the handpiece 10 further has a hollow cylindrical case 18 that covers a case 11. A suction tube 3c is connected to the proximal end 18a of the case 18. A sheath 21 is coupled to the distal end 18b of the case 18. A probe 30 is inserted in the sheath 21. In other words, the sheath 21 covers the probe 30 and is coupled to the distal end 18b of the case 18.

A space between the sheath 21 and probe 30 and between the case 11 and the case 18 defines a suction path 19. The suction path 19 communicates with the suction tube 3c at the proximal end 19a of the suction path 19. The middle part 19b of the suction path 19 lies between the sheath 21 and probe 30 and between the case 11 and the case 18. The distal end 19c of the suction path 19 has a suction port 19d. The suction port 19d is located at the distal end 21a of the sheath 21, lying between the distal end 21a and the probe 30. When the suction device 90 is driven, mist 2a is drawn into the suction path 19 through the suction port 19d, and flows through the suction tube 3c. Ultimately, the mist 2a is drawn into the suction device 90.

The ultrasonic transducer unit 12 of this embodiment is solid. The piezoelectric elements 12a are therefore shaped like discs in this embodiment.

The distal end 30a of the probe 30 projects from the distal end of the sheath 21.

Thus, the case 18 covers the case 11 and the space between the cases 11 and 18 defines the suction path 19 in the present embodiment. Therefore, the suction path 19 can have a larger cross section than in the first and second embodiments. The mist 2a can be drawn at a high rate, which can prevent the mist 2a from impairing the view field of the apparatus. Further, the mist 2a can be prevented from flashing due to the high-frequency current.

In this embodiment, the solid ultrasonic transducer unit 12 may be replaced by a solid ultrasonic transducer unit, because the suction path 19 is defined between the cases 11 and 18. Thus, the ultrasonic surgical instrument 1 according to this embodiment can acquire high versatility.

Moreover, the suction path 19 is not provided in the surgical element 31 as in the first embodiment. The present embodiment can use surgical elements of various shapes.

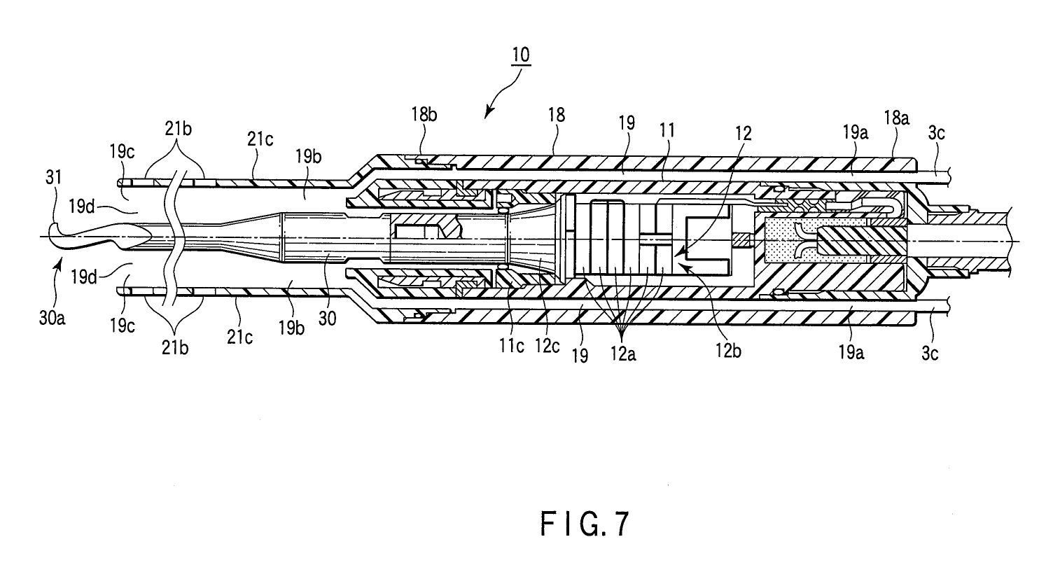

A fourth embodiment of the invention will be described with reference to FIG. 7. The components identical to those of the first to third embodiments are designated by the same reference numbers and will not be described.

At least one suction port 21b is made in the wall 21c of the sheath 21. In other words, the sheath 21 has at least one suction port 21b in its wall 21c. Like the suction port 19d, the suction port 21b communicates with the suction path 19 (more precisely, with the distal end 19c of the suction path 19).

In this embodiment, the suction port 19d and the suction port 21b cooperate to draw mist 2a at a higher rate than in the first to third embodiments, which can prevent the mist 2a from impairing the view field of the apparatus. Moreover, the mist 2a can be prevented from flashing due to the high-frequency current.

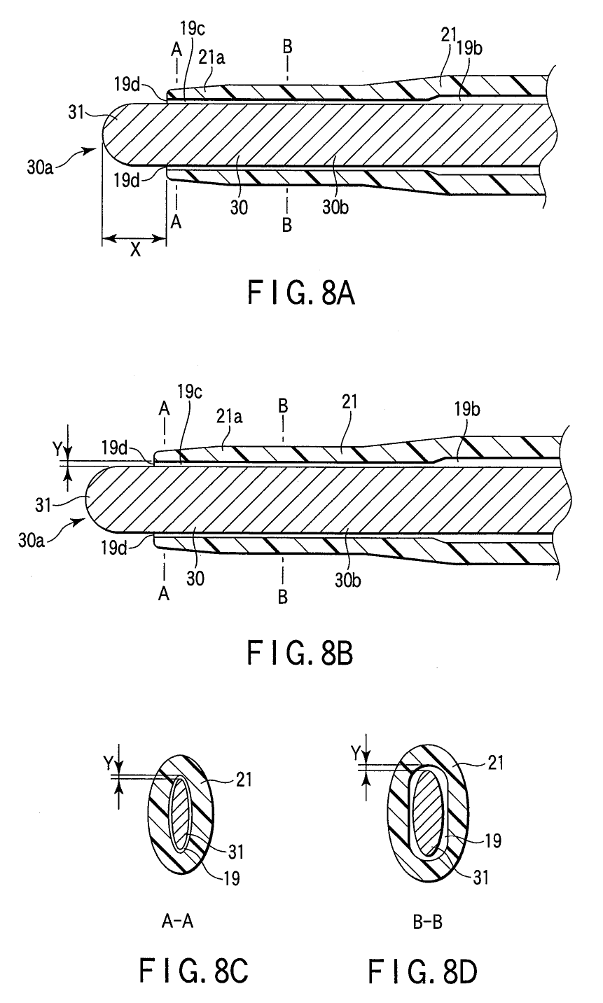

A fifth embodiment of the present invention will be described with reference to FIGS. 8A to 8D. The components identical to those of the first to fourth embodiments are designated by the same reference numbers and will not be described.

In the third and fourth embodiments, the distance X the distal end 30a projects from the distal end 21a as shown in FIG. 8A is about 3 to 5 mm. The distance X is of such a value for reducing the amount of mist 2a and for operating on the tissue 2 efficiently.

In the third and fourth embodiments, too, the gap Y between the probe 30 and the sheath 21, which is shown in FIG. 8B, is about 0.02 to 0.05 mm. The lower limit of the gap Y may be of such a value that the probe 30, even when vibrated, does not contact the sheath 21, not being damaged or not damaging the sheath 21.

As shown in FIGS. 8B, 8C and 8D, the gap Y at the distal end 30a (see the cross section of FIG. 8C, taken along line A-A in FIG. 8B) is narrower than the gap Y at the middle part 30b of the probe 30 (see the cross section of FIG. 8D, taken along line B-B in FIG. 8B). This helps to increase the suction pressure for drawing the mist 2a. For example, the gap Y at the distal end 30a is 0.02 mm, and the gap Y at the middle part 30b is 0.5 mm. Such gaps Y are also applied to the first and second embodiments.

As specified above, the distance X is as short as about 3 to 5 mm in this embodiment, reducing the area in which the probe 30 contacts the tissue 2. This minimizes the size of a piece of living tissue that may adhere to the probe 30, and can prevent the production of mist 2a.

In the present embodiment, the distance Y is shorter at the distal end 30a than at the middle part 30b. This raises the suction pressure, increasing the speed with which the mist 2a is drawn. The mist 2a can therefore be reliably drawn from the tissue 2.

In this embodiment, mist 2a is prevented from being produced and can be, if produced, drawn reliably. This can prevent the mist 2a from impairing the view field of the apparatus. Moreover, the mist 2a can be prevented from flashing due to the high-frequency current.

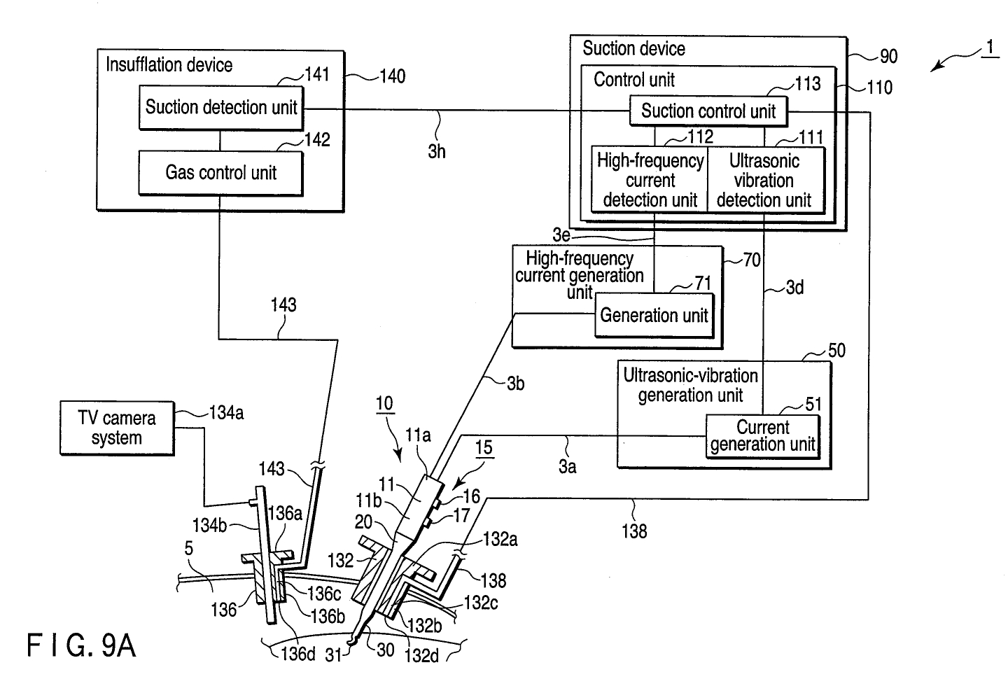

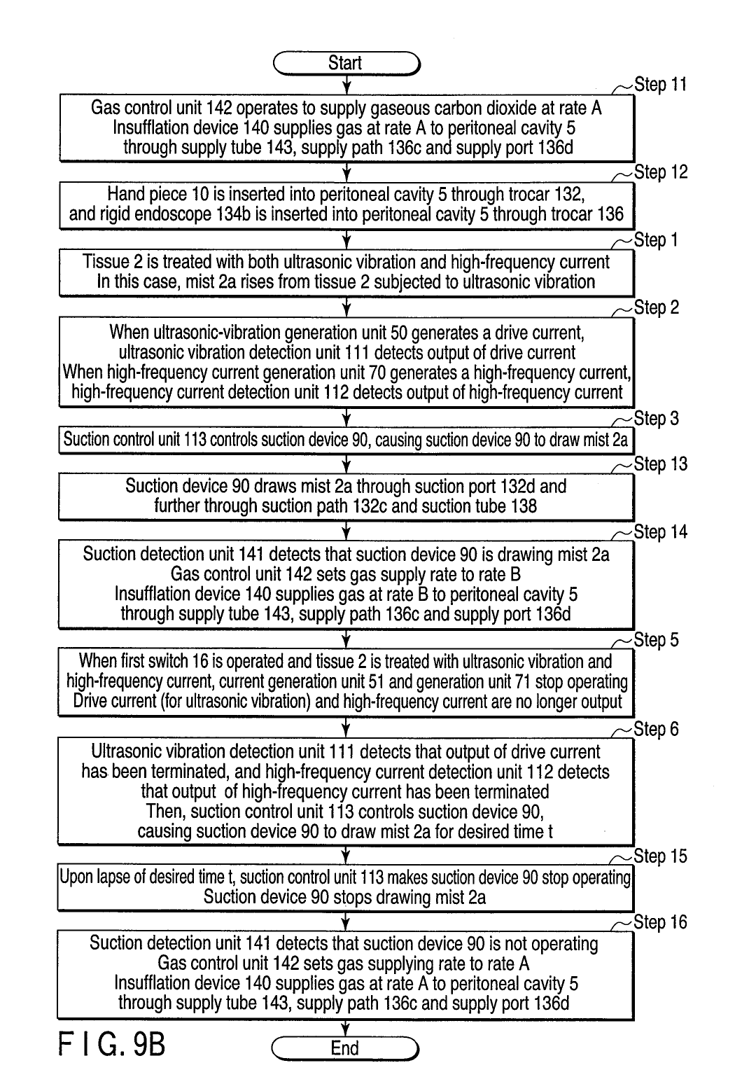

A sixth embodiment of this invention will be described with reference to FIGS. 9A and 9B. The components identical to those of the first to fifth embodiments are designated by the same reference numbers and will not be described. Note that the cable 3f and the counterelectrode 4 are not shown in FIGS. 9A and 9B.

As shown in FIG. 9A, an ultrasonic surgical instrument 1 according to this embodiment is designed for use in laparoscopy. The ultrasonic surgical instrument 1 has two trocars 132 and 136. Through the trocar 132, a surgical instrument such as a handpiece 10, may be inserted into the peritoneal cavity 5. Through the trocar 136, a rigid endoscope 134b connected to a TV camera system 134a may be inserted into the peritoneal cavity 5.

A suction tube 138 is connected at one end to the proximal end 132a of the trocar 132. The suction tube 138 is connected at the other end to the suction device 90.

The distal end 132b of the trocar 132 has a suction port 132d. The trocar 132 has a suction path 132c, through which the suction device 90 may draw mist 2a. The suction path 132c is coupled at one end to the suction port 132d and at the other end to the suction tube 138. The suction device 90 draws the mist 2a into the suction port 132c. The mist 2a thus drawn flows through the suction tube 138 into the suction device 90.

The handpiece 10 of this embodiment has neither suction paths 13 and 19 nor suction ports 13c and 19d.

To the proximal end 136a of the trocar 136 there is connected the supply tube 143 of an insufflation device (laparoscopy device) 140, which will be described later.

The distal end 136b of the trocar 136 has a supply port 136d. The trocar 136 has a supply path 136c through which the insufflation device 140 supplies gaseous carbon dioxide. The supply path 136c is coupled at one end to the supply port 136d and at the other end to supply tube 143. The insufflation device 140 supplies gaseous carbon dioxide from the supply tube 143 to the peritoneal cavity 5 through the supply path 136c and supply port 136d.

The ultrasonic surgical instrument 1 further has an insufflation device 140. The insufflation device 140 is coupled to the trocar 136 and configured to supply, for example, gaseous carbon dioxide to the insufflation device 140 through the trocar 136 when ultrasonic vibration and a high-frequency current are simultaneously output and the suction device 90 draws the mist 2a.

The insufflation device 140 is connected by a cable 3h to the suction device 90. The insufflation device 140 has a suction detection unit 141, a gas control unit 142, and a supply tube 143. The suction detection unit 141 detects the state in which the suction device 90 draws the mist 2a. The gas control unit 142 controls the rate of supplying gaseous carbon dioxide, in accordance with the rate of drawing the mist 2a, which has been detected by the suction detection unit 141. The supply tube 143 is provided to supply the carbon dioxide gas.

The suction detection unit 141 detects whether the suction device 90 is operating or not, from the operating state of the control unit 113. The “operating state” of the suction control unit 113 is whether or not the unit 113 makes (controls) the suction device 90 draw the mist 2a.

The gas control unit 142 controls the rate of supplying gaseous carbon dioxide, setting the difficult to a specific value before the ultrasonic vibration and the high-frequency current are simultaneously output, and to another specific value after the vibration and the current have been simultaneously output. More specifically, in the first mode, the gas control unit 142 makes rate A at which the gas is supplied before the vibration and the current are simultaneously output, lower than rate B at which the gas is supplied after the vibration and the current have been simultaneously output. In other words, the gas control unit 142 is configured to increases B at which the gas is supplied while the suction device 90 is drawing the mist 2a (while the ultrasonic vibration and the high-frequency current are being output), over the rate A at which the gas is supplied while the suction device 90 remains stopped (while the ultrasonic vibration and the high-frequency current are not output). The gas control unit 142 thus controls the rate of supplying carbon dioxide gas in the first mode. Rate A is, for example, about 1.5 L/min to about 10 L/min, while rate B is, for example, about 20 L/min to about 35 L/min. Rates A and B can be adjusted, each to a desired value, by operating, for example, a footswitch.

The gas control unit 142 sets rate B greater than rate A. The gas control unit 142 therefore prevents the suction device 90 from drawing the carbon dioxide gas supplied from the insufflation device 140. This prevents the pressure in the peritoneal cavity 5 from being lowered, and hence the peritoneal cavity 5 from being deflated. The TV camera system 134a can therefore preserve a sufficient view field. That is, rate B ensures a sufficient view field while the suction device 90 is being driven.

How the sixth embodiment operates will be explained with reference to the flowchart of FIG. 9B.

The gas control unit 142 operates to supply gaseous carbon dioxide at rate A. The insufflation device 140 supplies the gas at rate A to the peritoneal cavity 5 through the supply tube 143, supply path 136c and supply port 136d (Step 11).

The handpiece 10 is inserted into the peritoneal cavity 5 through the trocar 132, and the rigid endoscope 134b is inserted into the peritoneal cavity 5 through the trocar 136 (Step 12).

Next, Step 1 to Step 3 explained with reference to FIG. 3 are performed in sequence.

The suction device 90 draws the mist 2a through the suction port 132d and further through the suction path 132c and the suction tube 138. At this point, the suction device 90 also draws gaseous carbon dioxide (Step 13).

The suction detection unit 141 detects that the suction device 90 is drawing the mist 2a. The gas control unit 142 therefore sets the gas supply rate to rate B. As a result, the insufflation device 140 supplies the gas at rate B to the peritoneal cavity 5 through the supply tube 143, supply path 136c and supply port 136d (Step 14).

Thereafter, Step 5 and Step 6 (see FIG. 4) are performed.

Upon lapse of desired time t, the suction control unit 113 makes the suction device 90 stop operating. The suction device 90 therefore stops drawing the mist 2a (Step 15).

The suction detection unit 141 detects that the suction device 90 is not operating. Therefore, the gas control unit 142 sets the gas supply rate to rate A. As a result, the insufflation device 140 supplies the gas at rate A to the peritoneal cavity 5 through the supply tube 143, supply path 136c and supply port 136d (Step 16).

In this embodiment, the suction port 132d is made in the distal end 132b and the suction path 132c is provided in the trocar 132, as described above. The mist 2a can therefore be drawn, also achieving the same advantages in laparoscopy, as with the first embodiment. Further, the handpiece 10 can be simplified in configuration.

In this embodiment, the insufflation device 140 controls the rate of supplying gaseous carbon dioxide. This prevents the pressure in the peritoneal cavity 5 from being lowered, and hence the peritoneal cavity 5 from being deflated. The TV camera system 134a can therefore preserve a sufficient view field.

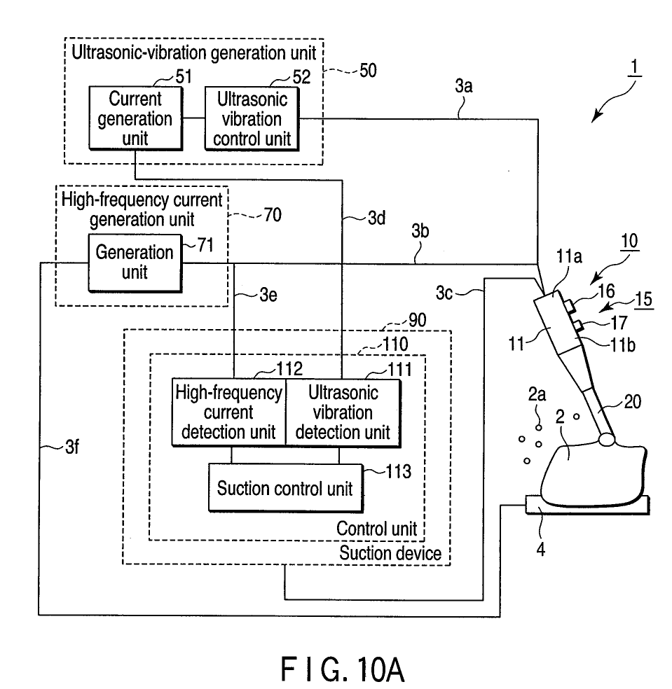

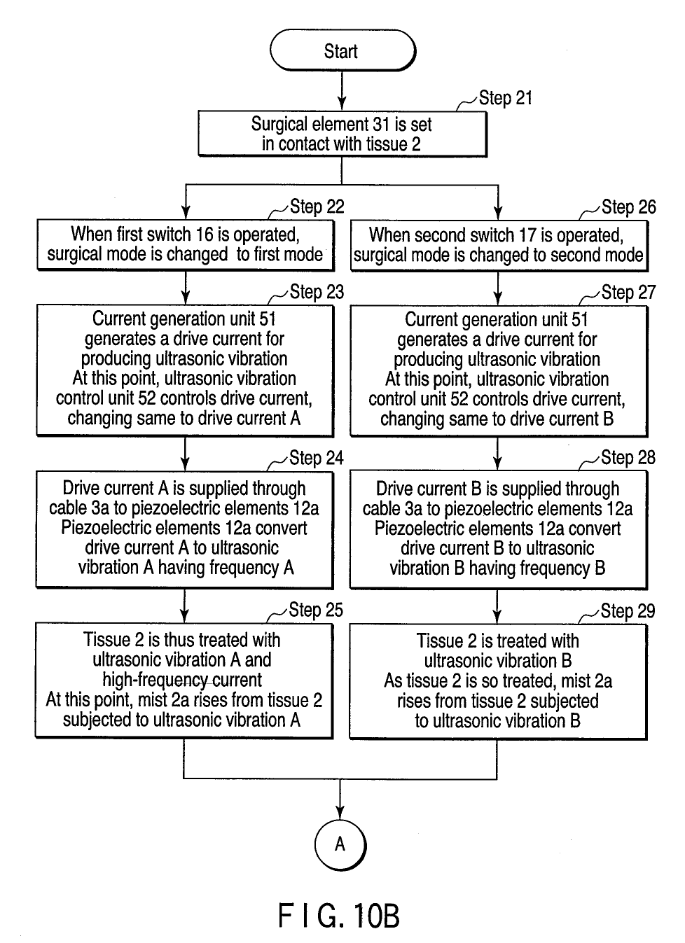

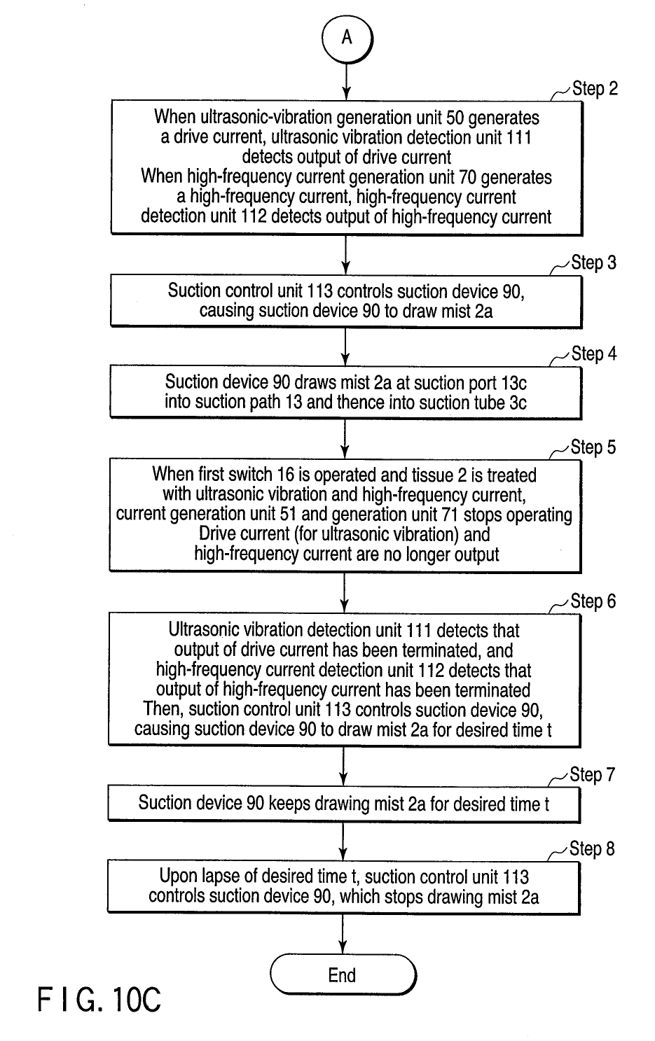

A seventh embodiment of the present invention will be described with reference to FIGS. 10A, 10B and 10C. The components identical to those of the first to sixth embodiments are designated by the same reference numbers and will not be described.

In the present embodiment, the ultrasonic-vibration generation unit 50 further has an ultrasonic vibration control unit 52, as is illustrated in FIG. 10A. The ultrasonic vibration control unit 52 is configured to control the drive current so that the frequency A of vibration in the first mode may be higher than the frequency B of vibration in the second mode. To be more specific, the ultrasonic vibration control unit 52 controls the frequencies A and B such that the frequency A becomes N times the frequency B, N being a natural number (N=2, 3, 4, . . . .) That is, the ultrasonic vibration control unit 52 controls the drive current A generated by the current generation unit 51 in the first mode, rendering it N times as large as the drive current B generated by the unit 51 in the second mode.

Generally, the higher the frequency of vibration, the smaller the cavitation will be. It is therefore required that the frequency A be higher than the frequency B in order to suppress the cavitation. The surgical element 31 is arranged at an antinode of the ultrasonic wave (where the vibration amplitude is the greatest) so that the surgical function of ultrasonic vibration may be utilized at most. Even if the vibration frequency (that is, the surgical mode) is changed, the frequency A is N times the frequency B that the surgical element 31 is arranged at an antinode of the ultrasonic wave. Note that a footswitch or the like may be operated so that the surgical element 31 may be arranged at an antinode of the ultrasonic wave and the frequency A may be set to N times the frequency B.

Frequency f (Hz) representing either the frequency A or B is a reciprocal of the wavelength λ; namely, f=1/λ. The surgical element 31 is located at an antinode of the ultrasonic wave having the length λ. In other words, the wavelength λ of ultrasonic vibration determines the overall length of the probe 30 (i.e., the position of the surgical element 31) in the present embodiment.

How this embodiment operates will be explained with reference to FIGS. 10B and 10C.

As shown in FIG. 10B, the surgical element 31 is set in contact with the tissue 2 to treat the tissue 2 (Step 21).

The surgeon operates the food switch, setting the vibration frequency to a desired value. The surgeon then operates the first switch 16, changing the surgical mode to the first mode (Step 22).

The current generation unit 51 therefore generates a drive current for producing ultrasonic vibration. At this point, the ultrasonic vibration control unit 52 controls the drive current, changing the same to drive current A (Step 23).

The drive current A is supplied through the cable 3a to the piezoelectric elements 12a. The piezoelectric elements 12a convert the drive current A to ultrasonic vibration A having frequency A (Step 24).

The ultrasonic vibration A is amplified by the horn 12c. The ultrasonic vibration A amplified is transmitted through the probe 30 to the surgical element 31. The surgical element 31 applies the vibration A to the tissue 2, which is thereby treated. As the tissue 2 is treated with the ultrasonic vibration A, mist 2a rises from the tissue 2.

In the first mode, the generation unit 71 generates a high-frequency current at the same time the current generation unit 51 therefore generates the drive current. The high-frequency current is supplied through the cable 3b to the piezoelectric elements 12a and thence to the surgical element 31 through the horn 12c and probe 30. The tissue 2 is thereby treated with the high-frequency current. After flowing from the surgical element 31 to the tissue 2, the high-frequency current flows to the counterelectrode 4 and back to the high-frequency current generation unit 70 through the cable 3f.

The tissue 2 is thus treated with the ultrasonic vibration A and high-frequency current. At this point, mist 2a rises from the tissue 2 subjected to ultrasonic vibration A (Step 25).

Subsequently, the operation goes to Step 2 shown in FIG. 10C.

The surgeon may operate the footswitch, setting the frequency to a desired value, and may then operate the second switch 17. In this case, the surgical mode is changed to the second mode (Step 26).

Then, the current generation unit 51 generates a drive current to producing ultrasonic vibration. At this point, the ultrasonic vibration control unit 52 controls the drive current, changing the same to drive current B (Step 27).

The drive current B is supplied through the cable 3a to the piezoelectric elements 12a. The piezoelectric elements 12a convert the drive current B to ultrasonic vibration B having frequency B (Step 28).

The ultrasonic vibration B is amplified by the horn 12c. The ultrasonic vibration B amplified is transmitted through the probe 30 to the surgical element 31. The surgical element 31 applies the vibration B to the tissue 2, which is thereby treated. As the tissue 2 is treated with the ultrasonic vibration B, mist 2a rises from the tissue 2 subjected to ultrasonic vibration B (Step 29).

Thereafter, the operation goes to Step S2 shown in FIG. 10C.

The sequence of operation, which follows Step 2 (FIG. 10C), is almost the same as Step 3 to Step 8 shown in FIG. 3, and will not be explained.

In the present embodiment, the vibration frequency can be controlled as the ultrasonic vibration control unit 52 controls the drive current, controlling the cavitation and, hence, the ultrasonic vibration. As a result, the production of mist 2a can be suppressed as the tissue 2 is treated with the ultrasonic vibration and the high-frequency current, and the mist 2a can be reliably drawn from the body cavity. This can prevent the mist 2a from impairing the view field of the apparatus. Moreover, the mist 2a can be prevented from flashing due to the high-frequency current. Moreover, the tissue 2 is treated with the ultrasonic vibration only, thus causing cavitation and drawing the mist 2a, ultimately ensuring a sufficient view field.

The present invention is not limited to the embodiments described above. The components of any embodiment can be modified in various manners in reducing the invention to practice at present, without departing from the sprit or scope of the invention. Further, the components of any embodiment described above may be combined, if necessary, in various ways to make different inventions.

For example, a footswitch (not shown) may be operated to change the surgical mode to the first mode or the second mode (not shown), not by operating the first switch 16 or the second switch as in any embodiment described above.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic surgical instrument comprising:
a handpiece configured to treat living tissue with at least one of an ultrasonic vibration and a high-frequency current;
a first generation unit configured to generate a drive current for producing the ultrasonic vibration;
a second generation unit configured to generate the high-frequency current;
a suction device configured to draw mist produced at the living tissue when the handpiece treats the living tissue with the ultrasonic vibration;
a control unit configured to control the suction device, causing the section device to draw the mist when outputs of the first generation unit and the second generation unit are detected or when only the output of the first generation unit is detected;
a suction tube connected to the suction device;
a suction path connected at a proximal end to the suction tube and configured to guide the mist drawn by the suction device; and
a suction port provided in a distal end of the suction path and configured to draw the mist into the suction path.

2. The ultrasonic surgical instrument according to claim 1, wherein the control unit controls the suction device, causing the suction device to draw the mist for a desired time when at least one of the outputs of the first generation unit and the second generation unit are detected after the first generation unit and the second generation unit have stopped outputting the ultrasonic vibration and the high-frequency current, respectively.

3. The ultrasonic surgical instrument according to claim 1, further comprising a handswitch configured to change a surgical mode in which to treat the living tissue with the handpiece, to a first mode in which the living tissue is treated with both the ultrasonic vibration and the high-frequency current or to a second mode in which the living tissue is treated with only the ultrasonic vibration.

4. The ultrasonic surgical instrument according to claim 1, wherein the handpiece has:
a first case;
an ultrasonic transducer unit provided in the first case and configured to convert the drive current generated by, and supplied, from the first generation unit, to the ultrasonic vibration;
a sheath arranged at a distal end of the first case;
a probe inserted in the sheath, connected at a proximal end to the ultrasonic transducer unit, and configured to transmit the ultrasonic vibration to a distal end; and
a surgical element arranged at the distal end of the probe, projecting from a distal end of the sheath and configured to treat the living tissue with the ultrasonic vibration produced by the first generation unit and transmitted from the probe, and the suction tube is connected to the proximal end of the first case,
the suction path is arranged, extending in a longitudinal direction of the handpiece, and connecting the ultrasonic transducer unit and the probe, and
the suction port is arranged in the surgical element.

5. The ultrasonic surgical instrument according to claim 1, wherein the suction device is a wall-embedded suction device.

6. The ultrasonic surgical instrument according to claim 1, the handpiece has:
a first case;
an ultrasonic transducer unit provided in the first case and configured to convert the drive current generated by, and supplied, from the first generation unit, to the ultrasonic vibration;
a second case covering the first case;
a sheath arranged at a distal end of the second case;
a probe inserted in the sheath, connected at a proximal end to the ultrasonic transducer unit, and configured to transmit the ultrasonic vibration to a distal end; and
a surgical element arranged at the distal end of the probe, projecting from a distal end of the sheath and configured to treat the living tissue with the ultrasonic vibration produced by the first generation unit and transmitted from the probe, and
the suction tube is connected to the proximal end of the second case,
the suction path is provided between the sheath and the probe and between the first case and the second case, and
the suction port is arranged in the distal end of the sheath and between the distal end of the sheath and the probe.

7. The ultrasonic surgical instrument according to claim 6, further having at least another suction port made in a wall of the sheath.

8. The ultrasonic surgical instrument according to claim 7, wherein a space between a distal end part of the probe and the sheath is narrower than a space between a middle part of the probe and the sheath.

9. The ultrasonic surgical instrument according to claim 1, further comprising:
a first trocar configured to guide the handpiece into a peritoneal cavity;
a second trocar configured to guide a rigid endoscope into the peritoneal cavity; and
an insufflation device configured to supply gaseous carbon dioxide into the peritoneal cavity through the second trocar, and
wherein the suction tube is connected at a proximal end to the first trocar, the suction path is arranged in the first trocar, and the suction port is provided in a distal end of the first trocar.

10. The ultrasonic surgical instrument according to claim 9, wherein the insufflation device is configured to supply the gaseous carbon dioxide at a higher rate while the suction device is drawing the mist than while the suction device remains stopped.

11. The ultrasonic surgical instrument according to claim 3, wherein the first generation unit further has an ultrasonic vibration control unit configured to control the drive current to set the ultrasonic vibration to a higher frequency in the first mode than in the second mode.

12. The ultrasonic surgical instrument according to claim 11, wherein the frequency of the ultrasonic vibration in the first mode is N times as high as the frequency of the ultrasonic vibration in the second mode, where N is a natural number.
